(19) United States
(12) Patent Application Publication
(10) Pub. No.: US 2020/0022656 A1
(43) Pub. Date: Jan. 23, 2020

(54) METHODS FOR DETECTION AND CONFIDENCE EVALUATION OF VITAL SIGN SIGNAL

(71) Applicant: Sil Radar Technology Inc., Kaohsiung City (TW)

(72) Inventors: Sheng-You Tian, Kaohsiung City (TW); Fu-Kang Wang, Kaohsiung City (TW)

(21) Appl. No.: 16/419,111

(22) Filed: May 22, 2019

(30) Foreign Application Priority Data
Jul. 18, 2018 (TW) 107124756

(51) Int. Cl. A61B 5/00; A61B 8/08; G06K 9/62

(52) U.S. Cl. CPC A61B 5/7221; A61B 5/0077; G06K 9/6214; A61B 8/08; A61B 5/7253; A61B 5/0075

(57) ABSTRACT

In a method for detection of vital sign signal, a variation detection module is configured to determine whether there is a living body in an image captured by an image capture module, next, a beam direction of a vital sign sensing system is directed toward the living body to detect vital sign signal of the living body, and a compute module is configured to compute eigenvalue and confidence of the vital sign signal so as to reconfirm whether the determined living body in the image is a living body having vital signs.

### FIELD OF THE INVENTION

This invention generally relates to methods for detection and confidence evaluation, and more particularly to methods for detection and confidence evaluation of vital sign signal.

### BACKGROUND OF THE INVENTION

With increased attention to health care, detection requirements for vital sign are increased. And noncontact vital sign sensing system has recently become a focal point because it has no restriction on installation site and has a longer detection distance than contact type vital sign sensing system.

The noncontact vital sign sensing system radiates wireless signals toward a part of human body and receives reflected signals from the part of human body. If the body part has a movement relative to the noncontact vital sign sensing system, the body movement may generate Doppler Effect on the wireless signals so that the reflected signals may contain Doppler shift components caused by the body movement. The noncontact vital sign sensing system can measure the body movement by demodulating the reflected signals, and the measured body movement is regarded as vital sign signals of human body while the body movement is caused by vital sign.

However, undesired movements, such as movements of other objects or other parts of human body, and noise signals may interfere with the noncontact vital sign sensing system to lead the reflected signals contain Doppler shift components caused by the undesired movements. Accordingly, it is difficult to identify the movements caused by vital signs of human body in the reflected signals and is possible to identify the undesired movements as the movements caused by vital signs.

### SUMMARY

The object of the present invention is to compute a confidence of a vital sign signal according a eigenvalue of the vital sign signal and a reference value by using a compute module, so that user can determine whether measured displacement information is the vital sign signal.

A method for detecting vital sign signal of the present invention comprises following steps: capturing an image by using an image capture module; determining whether there is a living body captured in the image by using a variation detection module; changing a beam direction of a vital sign sensing system toward the living body; detecting a vital sign signal of the living body by using the vital sign sensing system; receiving the vital sign signal from the vital sign sensing system and computing a eigenvalue of the vital sign signal by using a compute module; and evaluating a confidence of the vital sign signal according to the eigenvalue of the vital sign signal by using the compute module.

A method for evaluating confidence of vital sign signal of the present invention comprises following steps: detecting a vital sign signal of a living body by using a vital sign sensing system; receiving the vital sign signal from the vital sign sensing system and computing a eigenvalue of the vital sign signal by using a compute module; and evaluating a confidence of the vital sign signal according to the eigenvalue of the vital sign signal by using the compute module.

In the present invention, the variation detection module determines whether there is a living body captured in the image and the compute module computes the confidence of the vital sign signal to confirm whether the living body determined by the variation detection module is a living body having vital signs, so that the living body captured in the image is confirmed twice. By the vital sign sensing system, the vital signs of the living body captured in the image can be measured automatically.

### DESCRIPTION OF THE DRAWINGS

FIG. 1 is a flowchart illustrating a method for detection of vital sign signal in accordance with an embodiment of the present invention.

FIG. 2 is a functional block diagram illustrating a device using a method for detection of vital sign signal in accordance with an embodiment of the present invention.

FIG. 3 is a schematic diagram illustrating changing beam direction of a vital sign sensing system in accordance with an embodiment of the present invention.

FIG. 4 is a schematic diagram illustrating a vital sign sensing system in accordance with an embodiment of the present invention.

FIG. 5 is schematic diagram illustrating changing beam direction of a vital sign sensing system in accordance with an embodiment of the present invention.

### DETAILED DESCRIPTION OF THE INVENTION

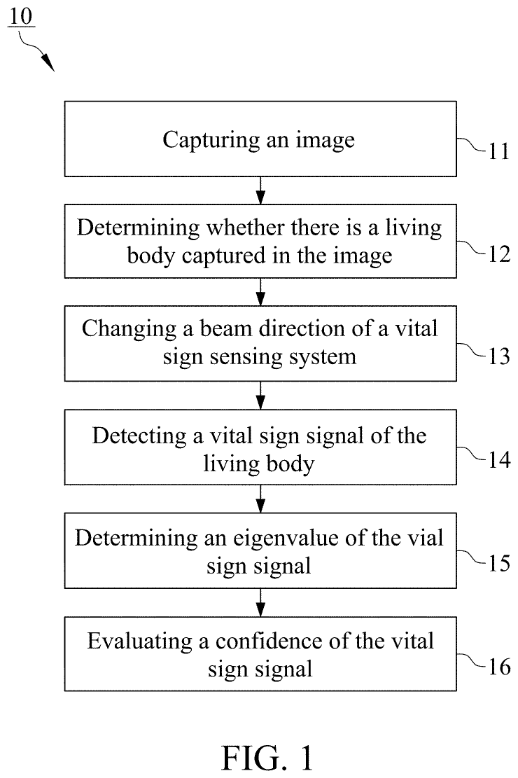

With reference to FIG. 1, a method 10 for detection of a vital sign signal in accordance with an embodiment of the present invention includes a step 11 of capturing an image, a step 12 of determining whether there is a living body captured in the image, a step 13 of changing a beam direction of a vital sign sensing system, a step 14 of detecting a vital sign signal of the living body, a step 15 of determining an eigenvalue of the vital sign signal and a step 16 of evaluating a confidence of the vital sign signal.

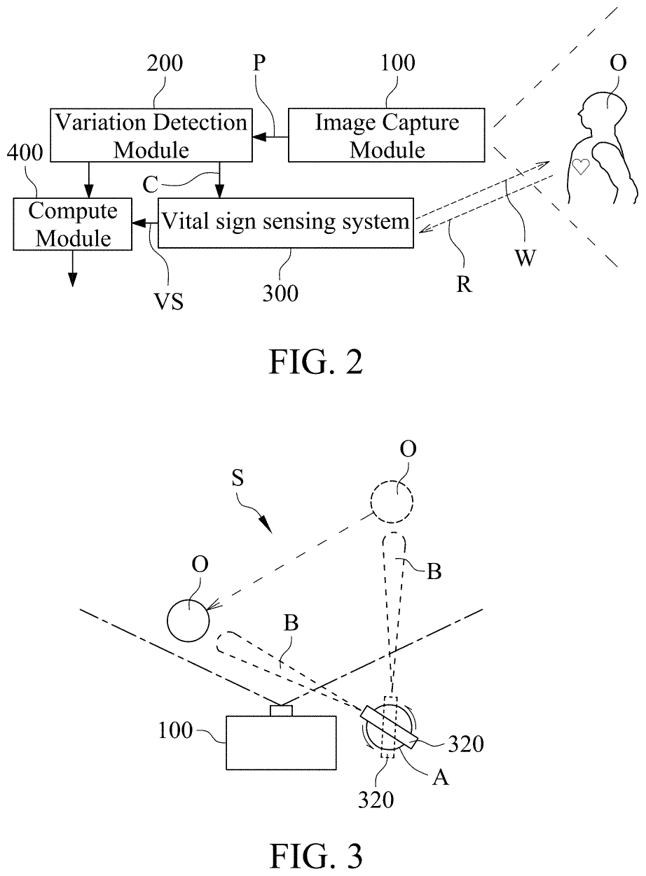

With reference to FIG. 2, a device using the method 10 to detect vital sign signal in this embodiment includes an image capture module 100, a variation detection module 200, a vital sign sensing system 300 and a compute module 400. The variation detection module 200 is electrically connected to the image capture module 100, the vital sign sensing system 300 is electrically connected to the variation detection module 200, the compute module 400 is electrically connected to the variation detection module 200 and the vital sign sensing system 300.

With reference to FIGS. 1, 2 and 3, the image capture module 100 is configured to capture an image P of a space S in the step 11. The image capture module 100 may be an optical camera, an infrared camera, a radar, an ultrasonic imaging system or a thermal imaging camera, and is capable to capture dynamic images.

With reference to FIGS. 1, 2 and 3, the image P captured by the image capture module 100 is transmitted to the variation detection module 200 which is configured to determine whether there is a living body O captured in the image P in the step 12. The image P changes over time so that the variation detection module 200 can compare each of pixels in the image P at different times to identify coordinates of the varied pixels in the images P. If coordinate displacements of the varied pixels are higher than a setting value, the variation detection module 200 determines the pixels having variations higher than the setting value are belong to the living body O. The setting value is determined according to the resolution of the image P and not limited in the present invention.

Further, the setting value may be a temperature range based on types of the target subject while the image P is a dynamic thermal image captured by a thermal imaging camera. For example, the temperature range may be between 32° C. and 40° C. when the target subject is human being, and the variation detection module 200 may determine the pixels belong to the living body O if the temperatures of the pixels in the image P are within the temperature range.

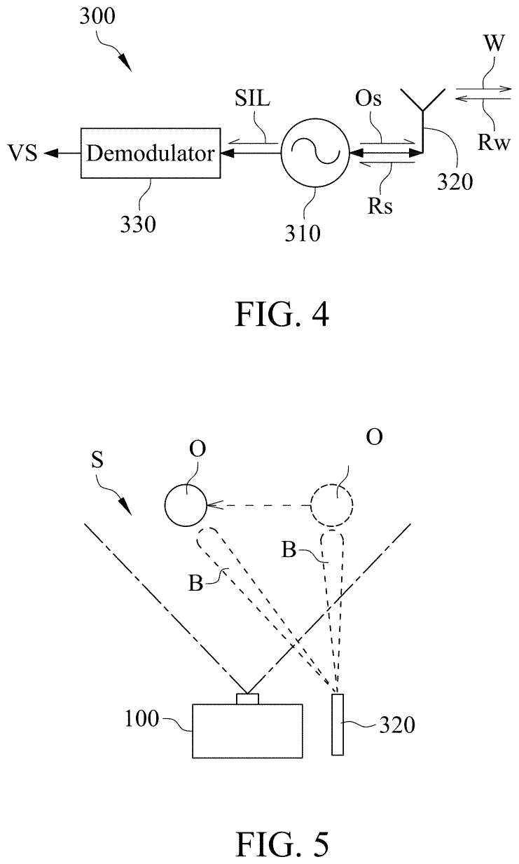

With reference to FIGS. 1 and 3, a beam direction B of the vital sign sensing system 300 is directed toward the living body O in the step 13. The vital sign sensing system 300 may be a continuous wave radar, a self-injection-locked radar or a pulse wave radar. Referring to FIGS. 2 and 4, the vital sign sensing system 300 in this embodiment is a self-injection-locked radar having extreme sensitivity to vital signs. The vital sign sensing system includes a self-injection-locked oscillator 310, an antenna 320 and a demodulator 330. The self-injection-locked oscillator 310 is configured to generate an oscillation signal Os. The antenna 320 is configured to receive the oscillation signal Os, radiate the oscillation signal Os as a wireless signal W toward the living body O and receive a reflected radio Rw from the living body O as a reflected signal Rs. A displacement of the living body O may cause Doppler effect on the wireless signal W such that the reflected radio Rw and the reflected signal Rs may contain Doppler shifts caused by the displacement of the living body O.

The reflected signal Rs received by the antenna 320 is transmitted and injected to the self-injection-locked oscillator 310 to make the self-injection-locked oscillator 310 operate in a self-injection-locked state and generate a self-injection-locked signal SIL. The reflected signal Rs contains the Doppler shifts caused by the displacement of the living body O and the frequency variation of the self-injection-locked signal SIL is proportional to the Doppler shifts caused by the displacement of the living body O, so that the displacement information of the living body O can be obtained by demodulating the self-injection-locked signal SIL.

As shown in FIG. 3, the antenna 320 may be a high directivity antenna and a beamwidth of the wireless signal W radiated from the antenna 320 is the beam direction B of the vital sign sensing system 300 such that the beam direction B is a fixed direction. The vital sign sensing system 300 in this embodiment further includes an actuator A which is configured to carry the antenna 320 and receive a control signal C from the variation detection module 200. The control signal C is used to control the actuator A to change the beam direction B of the vital sign sensing system 300. The antenna 320 of other embodiment, as shown in FIG. 5, may be an antenna array having multiple antennas and the phase of the wireless signal W from each of the antennas may be changed by the control signal C. The wireless signal W from each of the antennas may have direction-variable beam through beamforming method, the beam direction of the wireless signal W is the beam direction B of the vital sign sensing system 300 and the beam direction B is also directed toward the living body O. In this embodiment, after capturing the image P by using the image capture module 100 and determining whether the living body O is captured in the image P by using the variation detection module 200, the beam direction B of the vital sign sensing system 300 is changed to expand the monitoring range. Consequently, vital signs of multiple living bodies can be monitored by using a single vital sign sensing system 300 through changing the beam direction B.

Positions of the pixels determined as the living body O are identified according to an image coordinate of the image P, but the image coordinate is unavailable for identifying the position of the living body O when changing the beam direction B of the vital sign sensing system 300. As a result, the method 10 preferably further includes a step of transforming the image coordinate into a real coordinate by the variation detection module 200 before changing the beam direction B of the vital sign sensing system 300. The beam direction B of the vital sign sensing system 300 is able to be directed toward the living body O in accordance with the real coordinate.

With reference to FIGS. 1 and 2, the vital sign sensing system 300 is configured to detect a vital sign signal VS of the living body O in the step 14. Owing to the beam direction B of the vital sign sensing system 300 is directed toward the living body O in the step 13 and the self-injection-locked oscillator of the vital sign sensing system 300 is injection-locked by the reflected signal Rs, the demodulator 330 is able to receive the self-injection-locked signal SIL from the self-injection-locked oscillator 310 and demodulate the self-injection-locked signal SIL to obtain the vital sign signal VS of the living body O.

The variation detection module 200 in the step 12 only utilizes the image P to determine a moving object or an object having higher temperature as the living body O, but can't confirm whether the determined living body O is a living body having life characteristics. That is to say, the vital sign signal VS detected by the vital sign sensing system 300 may be displacement of a non-living body or noise from the space S, not vital signs of living body. Hence, the method 10 preferably further includes steps 15 and 16 used to determine whether the vital sign signal VS detected by the vital sign sensing system 300 is from vital signs of living body.

With reference to FIGS. 1 and 2, a compute module 400 is configured to receive the vital sign signal VS from the vital sign sensing system 300 to compute a eigenvalue of the vital sign signal VS in the step 15. The eigenvalue of the vital sign signal VS may be amplitude or phase of the vital sign signal VS for a period of time or a time point. The amplitude or phase computation is depending on the modulation method of the demodulator 330 of the vital sign sensing system 300.

While the demodulator 330 is a I/Q demodulator and the signal demodulated by the demodulator 330 has no DC offset component, the amplitude for a period of time is represented by

A=\sqrt{I²+Q²}

where I and Q are in-phase component and quadrature component of the vital sign signal VS, respectively.

While the demodulator 330 is a I/Q demodulator and the signal demodulated by the demodulator 330 has no DC offset component, different calculation formulas of the phase for a period of time are given by

$$φ = arctan ( \frac{Q}{I} )$$

$$φ = arctan ( \frac{I}{Q} )$$

$$φ = arctan ( - \frac{Q}{I} )$$

$$φ = arctan ( - \frac{I}{Q} )$$

one of the above calculation formulas of the phase for a period of time is selected according to circuit architectures and initial phase of the I/Q demodulator.

If the signal demodulated by the demodulator 330 contains DC offset component, the amplitude for a period of time is represented by

A=\sqrt{(I−DC_{I})²+(Q−DC_{Q})²}

where I is in-phase component of the vital sign signal VS and DC_{I} is DC offset of in-phase component of the vital sign signal VS.

If the signal demodulated by the demodulator 330 contains DC offset component, different calculation formulas of the phase for a period of time are represented by

$$φ = arctan ( \frac{Q - D {C}_{Q}}{I - D {C}_{I}} )$$

$$φ = arctan ( \frac{I - D {C}_{I}}{Q - D {C}_{Q}} )$$

$$φ = arctan ( - \frac{Q - D {C}_{Q}}{I - D {C}_{I}} )$$

$$φ = arctan ( - \frac{I - D {C}_{I}}{Q - D {C}_{Q}} )$$

one of the above calculation formulas of the phase for a period of time is selected according to circuit architectures and initial phase of the I/Q demodulator.

With reference to FIG. 1, the compute module 400 is configured to evaluate a confidence of the vital sign signal VS according to the eigenvalue of the vital sign signal VS and a reference value in the step 16. In this embodiment, a ratio between the eigenvalue of the vital sign signal VS and the reference value obtained by the compute module 400 is regarded as the confidence of the vital sign signal VS. For instance, the reference value may be a median or a constant of an amplitude of a reference vital sign signal for a period of time when the eigenvalue of the vital sign signal VS is the amplitude. In other embodiment, the reference value may be a mean value of a median or a constant of amplitudes of multiple reference vital sign signals for a period of time. The reference vital sign signals may be measured from males and females of different ages in advance.

The calculation formula of the confidence is given as follows:

$$C = \frac{A}{{A}_{t}} \times 100 \%$$

where C is the confidence of the vital sign signal VS and Aₜ is the reference value of the amplitude. The confidence is expressed in linear form so that the higher the calculated confidence value, the higher credibility the vital sign signal VS has.

In other embodiment, the calculation formula of the confidence may be represented as follows:

$$C = {W}^{\frac{A}{{A}_{t}}} \times 100 \%$$

where W is a constant and the confidence is expressed in exponential form.

Likewise, the reference value may be a median or a constant of the phase of the reference vital sign signal for a period of time when the eigenvalue of the vital sign signal VS is the phase.

In this embodiment, not only determine whether any living body O is captured in the image P by using the variation detection module 200, but also determine whether the living body O determined by the variation detection module 200 is a living body having vital signs according to the confidence obtained by the compute module 400, so that the living body O captured in the image P is confirmed twice. Accordingly, vital signs of living bodies in the space S can be detected automatically.

Not a single information of the vital sign signal VS, the eigenvalue in other embodiment may be a combination of multiple information, such as a combination of single information (amplitude or phase) of the time domain signal at multiple different time points, a combination of different information (amplitude and phase) of the time domain signal at same time point, a combination of different information of the time domain signal at multiple different time points, a combination of single information (amplitude or phase) of the frequency domain signal at multiple different frequencies, a combination of different information (amplitude and phase) of the frequency domain signal at same frequency, a combination of different information of the frequency domain signal at multiple different frequencies, or a combination of information of the time domain signal and the frequency domain signal. Like the eigenvalue, the reference value also may be an amplitude, a phase, a combination thereof or a weighting combination thereof of the time or frequency domain reference vital sign for a period of time or a time point.

The scope of the present invention is only limited by the following claims. Any alternation and modification without departing from the scope and spirit of the present invention will become apparent to those skilled in the art.

What is claimed is:

1. A method for detection of a vital sign signal, comprising:
capturing an image by using an image capture module;
determining whether there is a living body captured in the image by using a variation detection module;
changing a beam direction of a vital sign sensing system toward the living body;
detecting a vital sign signal of the living body by using the vital sign sensing system;
receiving the vital sign signal from the vital sign sensing system and computing a eigenvalue of the vital sign signal by using a compute module; and
evaluating a confidence of the vital sign signal according to the eigenvalue of the vital sign signal by using the compute module.

2. The method in accordance with claim 1, wherein the image capture module is an optical camera, an infrared camera, a radar, an ultrasonic imaging system or a thermal imaging camera.

3. The method in accordance with claim 1 further comprising a step of transforming an image coordinate of the image into a real coordinate by using the variation detection module before the step of changing the beam direction of the vital sign sensing system.

4. The method in accordance with claim 1, wherein the vital sign sensing system includes an actuator configured to change the beam direction of the vital sign sensing system.

5. The method in accordance with claim 1, wherein the vital sign sensing system includes a plurality of antennas configured to radiate a beam to change the beam direction through beamforming method.

6. The method in accordance with claim 1, wherein the eigenvalue of the vital sign signal is an amplitude, a phase or a combination thereof of the time domain vital sign signal or the frequency domain vital sign signal.

7. The method in accordance with claim 6, wherein the compute module is configured to compute an amplitude, a phase or a combination thereof of the time domain vital sign signal or the frequency domain vital sign signal for a period time or a time point as the eigenvalue of the vital sign signal.

8. The method in accordance with claim 1, wherein the compute module is to configured to compute a ratio between the eigenvalue of the vital sign signal and a reference value as the confidence of the vital sign signal.

9. The method in accordance with claim 6, wherein the compute module is configured to compute a ratio between the eigenvalue of the vital sign signal and a reference value as the confidence of the vital sign signal.

10. The method in accordance with claim 7, wherein the compute module is configured to compute a ratio between the eigenvalue of the vital sign signal and a reference value as the confidence of the vital sign signal.

11. The method in accordance with claim 8, wherein the reference value is an amplitude, a phase, a combination thereof or a weighting combination thereof of a time domain reference vital sign signal or a frequency domain reference vital sign signal for a period time or a time point.

12. The method in accordance with claim 10, wherein the reference value is an amplitude, a phase, a combination thereof or a weighting combination thereof of a time domain reference vital sign signal or a frequency domain reference vital sign signal for a period time or a time point.

13. A method for confidence evaluation of a vital sign signal, comprising:
detecting a vital sign signal of a living body by using a vital sign sensing system;
receiving the vital sign signal from the vital sign sensing system and computing a eigenvalue of the vital sign signal by using a compute module; and
evaluating a confidence of the vital sign signal according to the eigenvalue of the vital sign signal by using the compute module.

14. The method in accordance with claim 13, wherein the eigenvalue of the vital sign signal is an amplitude, a phase or a combination thereof of the time domain vital sign signal or the frequency domain vital sign signal.

15. The method in accordance with claim 14, wherein the compute module is configured to compute an amplitude, a phase or a combination thereof of the time domain vital sign signal or the frequency domain vital sign signal for a period time or a time point as the eigenvalue of the vital sign signal.

16. The method in accordance with claim 13, wherein the compute module is configured to compute a ratio between the eigenvalue of the vital sign signal and a reference value as the confidence of the vital sign signal.

17. The method in accordance with claim 14, wherein the compute module is configured to compute a ratio between the eigenvalue of the vital sign signal and a reference value as the confidence of the vital sign signal.

18. The method in accordance with claim 15, wherein the compute module is configured to compute a ratio between the eigenvalue of the vital sign signal and a reference value as the confidence of the vital sign signal.

19. The method in accordance with claim 16, wherein the reference value is an amplitude, a phase, a combination thereof or a weighting combination thereof of a time domain reference vital sign signal or a frequency domain reference vital sign signal for a period time or a time point.

20. The method in accordance with claim 18, wherein the reference value is an amplitude, a phase, a combination thereof or a weighting combination thereof of a time domain reference vital sign signal or a frequency domain reference vital sign signal for a period time or a time point.
